SEQUENCE LISTING (13 sequences)

SEQ ID NO: 1 - protein, 391 aa, Arabidopsis thaliana
MDVRESISLQNQVSMNLAKHVITTVSQNSNVIFSPASINVVLSIIAAGSAGATKDQILSFLKFSSTDQLNSFSSEIVSAVLADGSANGGPKLSVANGAWIDKSLSFKPSFKQLLEDSYKAASNQADFQSKAVEVIAEVNSWAEKETNGLITEVLPEGSADSMTKLIFANALYFKGTWNEKFDESLTQEGEFHLLDGNKVTAPFMTSKKKQYVSAYDGFKVLGLPYLQGQDKRQFSMYFYLPDANNGLSDLLDKIVSTPGFLDNHIPRRQVKVREFKIPKFKFSFGFDASNVLKGLGLTSPFSGEEGLTEMVESPEMGKNLCVSNIFHKACIEVNEEGTEAAAASAGVIKLRGLLMEEDEIDFVADHPFLLVVTENITGVVLFIGQVVDPLH

SEQ ID NO: 2 - protein, 392 aa, Brassica napus
MDVRESISLQNHVSLSLAKNVISTVSKNSNVIFSPASINVVLGIIAAGSTGETKDQILSFLNFPSIDQLNTFSSDIVSAVLADGSANGGPKLSAANAPWIDKSLSLQPSFKQLLDGSYKAASNLADFQTKAVEVIAEVNSWAEKETNGLITEVLPEGSADSMTRLIFANALYFKGTWNEKFDESLTKDGDFHLLDGSSKVTAPFMTSKKKQYVSAYDGFKVLGLPYLQGEDKRQFSMYLYLPDANNGLSDLLDKIVSTPGFLDSHIPRRQVKVGEFKIPKFKFSFGFEASDVLKGLGLASPFSGEDGLTEMVESPEMGKNLKVSSIFHKACIEVNEEGTEAAAASAGVIKLRGLAMEEEMIDFVADHPFLLVVMENITGVILFIGQVIDPLH

SEQ ID NO: 3 - protein, 4 aa, Artificial
  Reactive center loop
IKLA

SEQ ID NO: 4 - protein, 4 aa, Artificial
  Reactive center loop
VRPR

SEQ ID NO: 5 - protein, 4 aa, Artificial Sequence
  Reactive center loop
IRPR

SEQ ID NO: 6 - protein, 4 aa, Artificial Sequence
  Reactive center loop
WRHR

SEQ ID NO: 7 - protein, 4 aa, Artificial
  Reactive center loop
IKLK

SEQ ID NO: 8 - DNA, 17 bp, Artificial
  Forward primer
atggacgtgcgtgaatc

SEQ ID NO: 9 - DNA, 21 bp, Artificial
  Reverse primer
ttaatgcaacggatcaacaac

SEQ ID NO: 10 - DNA, 33 bp, Artificial
  Reverse mutagenic primer for IKLA
ctccataagcaatcctgctagctttataactcc

SEQ ID NO: 11 - DNA, 33 bp, Artificial
  Reverse mutagenic primer for IKLK
ctccataagcaatccttttagctttataactcc

SEQ ID NO: 12 - DNA, 39 bp, Artificial
  Reverse mutagenic primer for VRPR
cataagcaatcctcttggccttacaactccagctgatgc

SEQ ID NO: 13 - protein, 4 aa, Artificial Sequence
  Part of fluorometric substrate N-SUC-LLVY-4-Methylcoumaryl-7-amide
LLVY

(19) United States
(12) Patent Application Publication
(10) Pub. No.: US 2010/0333236 A1
(43) Pub. Date: Dec. 30, 2010

(54) INSECT INHIBITING PLANT SERPIN MUTANTS

(76) Inventors: Frank Van Breusegem, Gent (BE); Dominique Vercammen, Bottelare (BE); Guy Smagghe, Temse (BE); Fernando Alvarez, Regensdorf (CH)

Correspondence Address: TRASKBRITT, P.C., P.O. BOX 2550, SALT LAKE CITY, UT, 84110, US

(21) Appl. No.: 12/734,383

(22) PCT Filed: Oct. 30, 2008
(86) PCT No.: PCT/EP2008/064728

§ 371 Date: Sep. 3, 2010

Related U.S. Application Data
(60) Provisional application No. 61/001,218, filed on Oct. 30, 2007.

(30) Foreign Application Priority Data
Oct. 30, 2007 (EP) 07119602.6

(51) Int. Cl. A01H 5/00; A01N 37/18; A01P 7/02; A01P 7/04; C07K 14/415

(52) U.S. Cl. 800/298; 514/20.3; 530/370

(57) ABSTRACT

The present invention relates to a method to limit insect and/or mite damage in plants, by the use of mutant plant proteinase inhibitors. More specifically, it relates to the use of mutant serpins of which the reactive center loop has been replaced by an artificial sequence. Preferably, the mutant serpins use an Arabidopsis thaliana serpin-1 backbone, or a homologue thereof. The mutant serpins have a specificity other than the wild-type serpins, and by modulating the reactive loop center, specific inhibitors against specific insect and/or mite proteases can be developed. Those mutant serpins can be used to inhibit or limit insect and/or mite damage, such as the damage caused by insect feeding.

### CROSS-REFERENCE TO RELATED APPLICATIONS

This is a national phase entry under 35 U.S.C. §371 of International Patent Application PCT/EP/2008/064728, filed Oct. 30, 2008, published in English as International Patent Publication WO 2009/056597 A1 on May 7, 2009, which claims priority to European Patent Application Serial No. 07119602.6, filed Oct. 30, 2007, and to U.S. Provisional Patent Application Ser. No. 61/001,218, filed Oct. 30, 2007.

### TECHNICAL FIELD

The present invention relates to a method to limit insect and/or mite damage in plants, by the use of mutant plant proteinase inhibitors. More specifically, it relates to the use of mutant serpins of which the reactive center loop has been replaced by an artificial sequence. Preferably, the mutant serpins use an Arabidopsis thaliana serpin-1 backbone, or a homologue thereof. The mutant serpins have a specificity other than the wild-type serpins, and by modulating the reactive loop center, specific inhibitors against specific insect and/or mite proteases can be developed. Those mutant serpins can be used to inhibit or limit insect and/or mite damage, such as the damage caused by insect feeding.

### BACKGROUND

Insect pests are a serious problem in agriculture. They destroy millions of acres of staple crops such as corn, soybeans, peas, and cotton. Farmers must apply millions of liters of synthetic pesticides to combat these pests. However, synthetic pesticides pose many problems. They are expensive, force the emergence of insecticide-resistant pests, and they constitute a serious risk for the environment.

In order to develop a more economic and environmental pest control, biological approaches to pest control have been tried. In some cases, crop growers have introduced natural predators of the species sought to be controlled, such as non-native insects, fungi, and bacteria like Bacillus thurgiensis. Alternatively, crop growers have introduced large colonies of sterile insect pests in the hope that mating between the sterilized insects and fecund wild insects would decrease the insect population. However, none of these methods have been really successful.

As mentioned above, certain species of microorganisms of the genus Bacillus are known to possess pesticidal activity against a broad range of insect pests including Lepidoptera (Dulmage et al., 1981), Diptera (Goldberg and Margalit, 1977), Coleoptera (Krieg et al., 1987) and Hemiptera (Mathavan et al., 1987). Bacillus thuringiensis is probably the most successful biocontrol agents discovered to date. Pesticidal activity appears to be concentrated in parasporal crystalline protein inclusions, although insecticidal proteins have also been isolated from the vegetative growth stage of Bacillus. Several genes encoding these insecticidal proteins have been isolated and characterized. As a non-limiting example, such genes have been disclosed in WO9116432, WO9421795 and U.S. Pat. No. 5,366,892.

Microbial pesticides, particularly those obtained from Bacillus strains, have played an important role in agriculture as alternatives to chemical pest control. Insecticidal proteins isolated from strains of Bacillus thuringiensis, known as δ-endotoxins or Cry toxins, are initially produced in an inactive protoxin form. These protoxins are proteolytically converted into an active toxin through the action of proteases in the insect gut.

Once activated, the Cry toxin binds with high affinity to receptors on epithelial cells in the insect gut, thereby creating leakage channels in the cell membrane, lysis of the insect gut, and subsequent insect death through starvation and septicemia.

During recent years, agricultural scientists have developed crop plants with enhanced insect resistance by genetically engineering crop plants to produce insecticidal proteins from Bacillus. Such plants have been disclosed in, amongst others, WO9116432 and WO9421795. However, these Bt insecticidal proteins only protect plants from a relatively narrow range of pests. Thus, there is an immediate need for methods that enhance the effects of insecticidal proteins.

One possibility to enhance the insect resistance is the co-expression of the Bt insecticidal proteins with another polypeptide having insecticidal activity, as disclosed in WO2005083095. Alternatively, the application of other broad spectrum insect inhibitory polypeptides, such as proteinase inhibitors, has been studied. An overview of proteinase inhibitor genes used in combat against insects, pests and pathogens is given by Haq et al. (2004). However, thr pH dependency and specificity of the proteinase inhibitor is extremely important and not all proteinases inhibitors are successful. In some cases, the transgenic plants overexpressing the proteinase inhibitor caused a growth stimulation of the larvae (Girard et al., 1998), although the inhibitor used was capable of inhibiting the digestive proteases of the larvae. This indicates that a selection of a proteinase inhibitor purely on in vitro characteristics is very difficult, if not impossible.

Within the group of proteinase inhibitors, plant proteinase inhibitors and within this group, serpins have drawn some interest as possible pest inhibitors. Plant serpins have been studied intensively over the past years. In cereals, serpins are collectively called Z proteins, and constitute 5% of the total albumin in grains (Hejgaard, 1982). Several serpins from wheat, barley, rye, and oat have been identified and characterized (Rasmussen, 1993; Østergaard et al., 2000; Brandt et al., 1990; Lungaard and Svensson, 1989; Rosenkrands et al. 1994; Rasmussen et al., 1996; Dahl et al., 1996; Hejgaard, 2001; Hejgaard and Hauge, 2002). Biochemical studies with plant serpins from cereals have demonstrated their inhibitory action against different animal proteases such as trypsin, chymotrypsin, cathepsin G, and elastase (Østergaard et al., 2000; Dahl et al., 1996; Hejgaard, 2001; Hejgaard and Hauge, 2002). Also, the serpin isolated from squash phloem was reported to be an elastase inhibitor (Yoo et al., 2000).

Whereas in animals, serpins are known to be involved in fundamental biological processes (Patston, 2000; van Gent et al., 2003), no precise role has yet been assigned to plant serpins. The high abundance of serpins in cereal seeds has led to the hypothesis that they could be involved in defense of storage tissue against insect feeding (Østergaard et al., 2000; Hejgaard and Hauge, 2002). Although direct feeding of aphids with CmPS-1 did not affect their survival, the negative correlation between the survival of aphids feeding on squash leaves and expression levels of the phloem serpin CmPS-1 supports the defense theory (Yoo et al., 2000). Gene expression studies in barley have revealed that serpin transcripts are not only present in developing grain, but also in vegetative tissues, such as roots, shoots, and leaves. Serpin protein could be detected in phloem cells, meristem, and root cap cells of the young root, and in root cap, coleorhiza, and apical meristem of embryonic roots. Also in young leaves, some phloem cells produced serpin proteins (Roberts et al., 2003). Although these expression patterns are intriguing, complete insight into the precise functional role of plant serpins is still lacking.

Based on this possible protection, several serpins havebeen tested as insect inhibitors, but none of those have been extremely effective. WO9413810 discloses the use of a Nicotania alata type II serine proteinase inhibitor precursor with at least four domains for increasing plant resistance to pest, but the effect is limited to a retardation in growth of the nymphs tested. US2003/0018990 discloses the use of a serine proteinase of Brassica oleracea for obtaining resistance to herbivorous insects. Depending upon the parameters measured and the insects used, the results obtained are comparable or less efficient than the results obtained with Bt. Yoo et al. (2000) described the use of Cucurbita maxima phloem serpin-1 for inhibiting the piercing-sucking aphid Myzus persicae. Although they claim to see a decrease in survival on the transgenic plants in function of the time, they could not demonstrate any toxicity when using the phloem and the effects of survival on the transgenic plants might be due to other factors.

The reactive center loop (RCL) of serpins is essential for their specificity. The “reactive center loop,” as defined here, is defined by the P1 position after which the target proteinase is cleaving the proteinase inhibitor. The RCL as defined here encompasses at least four amino acids, preferably five amino acids, even more preferably, six amino acids, including the P1 and aminoterminal of the cleavage site, i.e., P1 is the carboxyterminal amino acid after cleavage. A “mutation,” as used here, is any change in amino acid in this RCL. Preferably, it is a non-conservative change of one or more amine acids. Mutations in the RCL are supposed to block the activity of the serpin, as the RCL acts as substrate for the protease and exerts its inhibiting activity by competition with the natural substrate. Indeed, Vercammen et al. (2006) demonstrated that mutation of the RCL loop into an IKLA (SEQ ID NO:3) or a VRPR (SEQ ID NO:4) sequence resulted in a significant loss of inhibitory activity.

Surprisingly, we found that those mutant serpins do have an inhibiting activity against insect proteases, and that this activity may be far more pronounced than the activity of the wild-type serpin. Mutations of the RCL, without—or with limited—changes in the backbone of the serpin, allows design of insect-specific serpins, and opens the possibility to design plants with a specific resistance against some insects, without affecting non-target organisms.

### DISCLOSURE

A first aspect of the invention is the use of a recombinant serpin, comprising a mutant reactive center loop, for limiting insect- and/or mite-provoked damage in plants. A “mutant RCL” as used herein means that the RCL differs from the sequence that occurs in the corresponding wild-type serpin. Preferably, the serpin belongs to the inhibitor family IA, as determined by MEROPS (http://merops.sanger.ac.uk), showing sequence homology to human alpha1-antitrypsin. Preferably, the serpin is selected from the group consisting of Arabidopsis thaliana serpin At1g47710, Barley serpin BZx, Barley serpin BZ7, Wheat serpin WZ2b, Cucumis sativus serpin gi|58416137, Oryza sativa serpin gi|37700305, Citrus x paradise serpin gi|26224736, Solanum tuberosum serpin gi|62950609, Lycopersicon esculentum serpin gi|14685955, Nicotiana tabacum serpin gi|52837819, Brassica rapa serpin gi|54416864, Vitis vinifera serpin gi/33401535, Antirrhinus majus serpin gi|51113970, Triphysaria versicolor serpin gi|68033793, Helianthus paradoxus serpin gi|33123072, Gossypium arboreturni serpin gi|13245345, Populus nigra serpin gi|60218046 and Brassica napus serpin (SEQ ID NO:2). Even more preferably, the backbone of the serpin is derived from SEQ ID NO:1 (At1g47710). Preferably, the mutant serpin comprises a RCL comprising a sequence selected from the group consisting of IKLA (SEQ ID NO:3), VRPR (SEQ ID NO:4), IRPR (SEQ ID NO:5), WRHR (SEQ ID NO:6) and IKLK (SEQ ID NO:7). Even more preferably, the mutant serpin comprises a RCL comprising IKLA (SEQ ID NO:3).

Preferably, the insect- and/or mite-caused damage is caused by a chewing insect. More preferably, the insect or mite belongs to an order selected from the group consisting of the orders Lepidoptera, Coleoptera, Homoptera and Acari. Even more preferably, the insect or mite is selected from the group consisting of Spodoptera spp, Sesamia spp., Ostrinia spp., Leptinotarsa spp., Tribolium spp., Acyrthosiphon spp., Bemisia spp. and Tetranychus spp. Most preferably, the insects or mites belong to the species Spodoptera littoralis, Sesamia nonagroides, Ostrinia nubilalis, Leptinotarsa decemlineata, Tribolium castaneum, Acyrthosiphon pisum, Bemisia tabaci and/or Tetranychus urticae.

“Insect damage,” as used here, can be any damage such as visual damage to leaf, stem, seeds or fruit, or a decrease in yield, when compared with the plant that is not affected by insects or mites. Preferably, the damage is a decrease in yield. Limiting insect damage means that the damage in the treated plant is less severe than in the non-treated control.

A preferred embodiment is the use of the serpin, whereby the serpin is applied by spraying. Another preferred embodiment is the use of the serpin, whereby the serpin is overexpressed in a plant. In the case of overexpressing, the concentration in the plant material is preferably at least 0.65 mg/kg wet weight, even more preferably, at least 6.5 mg/kg wet weight, and most preferably, at least 65 mg/kg wet weight.

Still another aspect of the invention is a method to develop new mutant serpins with a specific inhibition spectrum, comprising the mutation of one or more amino acids of the RCL. A “specific inhibition spectrum” as used here means the mutant serpin inhibits other proteinases than the wild-type serpin, or shows a significantly higher inhibition towards a proteinase that is inhibited by the wild-type serpin. “Significantly higher” as used here means that the inhibition occurs at significantly lower concentration of the serpin, and/or that a significantly higher inhibition percentage is reached. Preferably, a specific inhibition spectrum means that other proteinases are inhibited than by the wild-type mutant. Even more preferably, by the change in inhibition spectrum, proteinases can be inhibited that are not inhibited by the wild-type serpin, when both mutant and wild-type serpin are used at the same concentration.

Another aspect of the invention is a transgenic plant, expressing a recombinant serpin as comprised according to the invention. Preferably, the plant is used to limit insect damage caused by insects feeding on the plant.

### BRIEF DESCRIPTION OF THE FIGURES

FIG. 1: Inhibition of serine-like protease activities from S. littoralis extracts by AtSerpin1 (WT) and its derived variant IKLA (SEQ ID NO:3). Data are the mean±SE of duplicate measurements from a unique pool of gut extracts.

FIG. 2: Inhibition of serine-like protease activities from S. nonagrioides extracts by AtSerpin1 (WT) and its derived variant IKLA (SEQ ID NO:3). Data are the mean±SE of duplicate measurements from a unique pool of gut extracts.

FIG. 3: Inhibition of cysteine-like protease activities from L. decemlineata extracts by AtSerpin1 (WT) and its derived variant IKLA (SEQ ID NO:3). Data are the mean±SE of duplicate measurements from a unique pool of gut extracts.

FIG. 4: Inhibition of cysteine-like protease activities from T castaneum extracts by AtSerpin1 (WT) and its derived variant IKLA (SEQ ID NO:3). Data are the mean±SE of duplicate measurements from a unique pool of gut extracts.

FIG. 5: Inhibition of cysteine-like protease activities from A. pisum extracts by AtSerpin1 (WT) and its derived variant IKLA (SEQ ID NO:3). Data are the mean±SE of duplicate measurements from a unique pool of gut extracts.

FIG. 6: Inhibition of cysteine-like cathepsin-L protease activities from B. tabaci extracts by AtSerpin1 (WT) and its derived variant IKLA (SEQ ID NO:3). Data are the mean±SE of duplicate measurements from a unique pool of gut extracts.

FIG. 7: Inhibition of cysteine-like protease activities from T. urticae extracts by AtSerpin1 (WT) and its derived variant IKLA (SEQ ID NO:3). Data are the mean±SE of duplicate measurements from a unique pool of gut extracts.

FIG. 8A: Micrograph showing a first-instar nymph of A. pisum treated for two days with 10000 μg/ml serpin mutant IKLA (SEQ ID NO:3). In intoxicated aphid nymphs, clear symptoms were observed of inhibited growth as compared to control aphids (control) with a significant reduction in aphid size (the aphid size was reduced about 50%) and then followed by aphid mortality (the dead aphid colors brown).

FIG. 8B: Dose-response curve for percent mortality of the aphid Acyrthosiphon pisum when fed for three days on an artificial diet comprising the AtSerpin1 mutant IKLA (SEQ ID NO:3). The estimated median lethal concentration (LC50) was 406 μg/ml with a 95% confidence interval of 271-607 μg/ml (Logarithm (LC50)=2.608±0.0785; R2=0.95).

### DETAILED DESCRIPTION OF THE INVENTION

### Examples

### Materials and Methods to the Examples

### Recombinant Atserpin1 Production; Construction and Production of the Mutants

Recombinant Atserpin1 production and purification was done as described in Vercammen et al., 2006.

The cDNA for the ORF of At1g47710 was obtained by RT-PCR with the following forward and reverse primers, provided with the adequate 5′ extensions for Gateway™ cloning (Invitrogen): 5′-ATGGACGTGCGTGAATC-3′ (SEQ ID NO:8) and 5′-TTAATGCAACGGATCAACAAC-3′ (SEQ ID NO:9). After recombination in pDEST17, the plasmid was introduced into E. coli strain BL21(DE3)pLysE and production of the HIS6-tagged protein induced by incubation in 0.2 mM isopropyl-β-D-thiogalactopyranoside for 24 hours. The protein was purified by metal ion affinity chromatography (TALON™; BD, Franklin Lakes, N.J.). Protein concentration and purity were checked by Bradford analysis (BioRAD) and SDS-polyacrylamide gel eletrophoresis (PAGE). Point mutagenesis of AtSerpin1 was done using the “megaprimer” method. The reverse mutagenic primers used were:

IKLA (SEQ ID NO: 3),
(SEQ ID NO: 10)
5′-CTCCATAAGCAATCCTGCTAGCTTTATAACTCC-3′;
IKLK (SEQ ID NO: 7),
(SEQ ID NO: 11)
5′-CTCCATAAGCAATCCTTTTTAGCTTTATAACTCC-3′;
and
VRPR (SEQ ID NO: 4),
(SEQ ID NO: 12)
5′-CATAAGCAATCCTCTTGGCCTTACAACTCCAGCTGATGC-3′.

Bacterial production and purification were performed as described for wild-type AtSerpin1.

### Insects

A selection of economically important pest insects was made to support the insecticide potency and wide target pest range of AtSerpins. The selected Lepidoptera (caterpillars) and Coleoptera (beetles) are representative pest insects with biting-chewing mouth paths and important in agriculture, horticulture, forestry and stored products. The selected Homoptera (aphids, whiteflies) and Acari (mites) are good representatives for piercing-sucking pests, being very important in agriculture, horticulture, forestry.

Lepidoptera:

A colony of the cotton leafworm Spodoptera littoralis (CLW) (Lepidoptera: Noctuidae) was reared on a semi-artificial diet at standard conditions in the laboratory. Larvae were collected and frozen at −20° C. until analysis.

For the artificial feeding tests, newly moulted (zero to six hours) third instar larvae of the cotton leafworm Spodoptera littoralis (Lepidoptera: Noctuidae) (CLW) were selected from a continuous stock colony in the Laboratory of Agrozoology at Ghent University, Belgium, that was kept at standard conditions of 23±2° C.; 65±5% relative humidity and a 16:8 (light:dark) regime (Smagghe et al., 2002).

The cotton leafworm is a polyphagous noctuid species of world economic importance in agriculture and horticulture. Such caterpillars cause high levels of damage in at least 87 crop species belonging to 40 families distributed all over the world. Also, many populations developed high levels of resistance towards most insecticide groups (Alford, 2000).

Larvae of Mediterranean corn borer, Sesamia nonagroides (MCB) (Lepidoptera: Noctuidae), were frozen and stored at −20° C. until needed.

Larvae of European corn borer, Ostrinia nubilalis (Lepidoptera: Crambidae) (ECB), were collected and frozen at −20° C. until analysis.

Coleoptera:

Colorado potato beetles Leptinotarsa decemlineata (CPB) (Coleoptera: Chrysomelidae) were reared on freshly cut potato foliage, Solanum tuberosum, collected and frozen at −20° C. until analysis.

Adults of the red flour beetle, Tribolium castaneum (Coleoptera: Tenebrionidae) were selected from a colony maintained in the laboratory at standard conditions, frozen and stored at −20° C. until needed.

Homoptera:

A laboratory colony of the pea aphid Acyrthosiphon pisum (PA) (Homoptera: Aphididae) was reared on pea plants, Vicia faba. Adults were selected, frozen and stored at −20° C. until needed.

Green peach aphid adults, Myzus persicae (Homoptera: Aphididae) were selected, frozen and stored at −20° C. until needed.

Adults of the sweetpotato whitefly, Bemisia tabaci (Homoptera: Aleyrodidae) were selected, frozen and stored at −20° C. until needed.

Acari:

Spider mites, Tetranychus urticae (Acari: Tetranychidae) were selected from a colony maintained in the laboratory at standard conditions, frozen and stored at −20° C. until needed.

Inhibitory Activity of Arabidopsis thaliana Serpins

Complete guts from CLW, MCB, ECB and CPB larvae were dissected and subsequently homogenized in 0.15 M Nacl, centerfuged at 10,000 g for five minutes, and the supernatants pooled and stored frozen (−20° C.). FOr PA, whole insect bodies were used.

Inhibitory activity of wild-type Serpin1 from Arabidopsis thaliana and its derived variant IKLA (SEQ ID NO:3) were tested in vitro against serine-like protease activities from CLW, MCB (trypsin and chymotrypsin) and ECB (trypsin and elastase), and against cysteine-like protease activities (cathepsin-B and cathepsin-L) from PA and CPB. A. thaliana serpins were preincubated at room temperature with the gut extracts for 15 minutes, prior to addition of substrate. Serpin concentrations varied from 0.02-20 μM. Standard fluorometric substrates used were Z-Arg-Arg-amc, Z-Phe-Arg-amc, and N-Suc-Leu-Leu-Val-Tyr-4-Methylcoumaryl-7-amide (SEQ ID NO:13), from Bachem. For measuring elastase activity, the colorimetric substrate Sa2PppNa (N-succinyl-(alanine)2-prolinephenylalanin-p-nitroanilide) were used, from Sigma. All assays were carried out in duplicate with pooled gut extracts. Fluorescence was monitored for 30 minutes at λex of 380 nm and λem of 460 nm in a microtiter plate reader. For measuring the elastase activity in ECB, the colorimetric substrate Sa2PppNa (N-succinyl-(alanine)2-proline-phenylalanin-p-nitroanilide) were used, from Sigma.

Absorbance was measured at 410 nm for elastase activity in a microplate reader. Insect protease activities were determined at 30° C., at pH 10.5 for CLW, MCB and ECB, and at pH 7.5 for PA and CPB, in 100 μl of reaction mixture.

Inhibitory activity of the derived variants IKLK (SEQ ID NO:7) and VRPR (SEQ ID NO:4) were also tested in vitro against trypsin activity of CLW and cathepsin B activity of PA. Standard colorimetric substrates used were BApNa (Na-benzoyl-DL-arginine-p-nitroanilide) and ZAA2MNA (N-carbobenzoxyalanine-alanine-arginine 4-methoxy-β-naphthyl amide), from Sigma. All assays were carried out in triplicate with pooled gut extracts. Serpin concentrations were 5 μM and 10 μM. Absorbance was measured at 410 nm for trypsin activity and 520 nm for cathepsin B activity in a microplate reader. Insect protease activities were determined at 30° C., at pH 10.5 for CLW and at pH 7.5 for PA, in 100 μA of reaction mixture.

Insect Bioassay with Pea Aphid in a Feeding Apparatus with Artificial Assay Supplemented with Recombinant Serpin Mutant IKLA (SEQ ID NO:3) Protein.

A colony of the pea aphid (Acrythosiphon pisum; Homoptera: Aphididate) was maintained continuously at the Laboratory of Agrozoology (Ghent University) on young Vica faba L. plants that are grown in a plant growth room at 23° C. to 25° C., 60% to 65% relative humidity, and a photoperiod of 16 hours light:8 hours dark. Mature aphids are put on plants for 24 hours and the resulting offspring nymphs (aged zero to 24 hours) are used in the insect bioassay experiments.

As food for the aphids, a standard diet previously developed for A. pisum (Febvay et al., 1988) was used as the basal diet to which the recombinant serpin IKLA (SEQ ID NO:3) mutant protein was added. Then the prepared liquid artificial diet was filter-sterilized through a 0.2-μm filter (Millipore Corp., Bedford, Mass., USA). Aliquots of 1.5 ml can be stored in the freezer at −20° C. for a period up to six months. After defrosting at room temperature, the artificial diet is ready to be used.

The feeding apparatus was prepared in-house using plexiglass cylinders (3 cm high and 3 cm diameter). The food sachet was made under sterile conditions and consists of two layers of parafilm membrane on top of the container. 200 μl of the artificial diet was sandwiched between the two layers.

To challenge aphids to recombinant serpin protein, a series of concentrations (100-10000 μg/ml) of the protein (made in PBS buffer) was prepared in the artificial diet. In the treatments, 200 μl of each concentration was used to make a food sachet. In the controls, the diet was supplemented with a corresponding amount of buffer. Per sucking apparatus, 15 synchronized neonate nymphs (aged zero to 24 hours) were transferred onto the artificial diet. For each concentration, three replicates were carried out, and the experiment was performed two times independently from each other; a total of 90 aphids were tested per concentration. Aphids were checked daily during three consecutive days for dead individuals to calculate mortality percentages.

Differences between treatments were considered significant at p=0.05. The median lethal concentration 50% (LC₅₀) and the 95% confidence interval were determined from the dose-response curve using the nonlinear regression analysis in Prism v4 (GraphPad, San Diego, Calif., USA) (Smagghe and Degheele, 1994).

### Example 1

### Inhibition of Enzymes of the Cotton Leafworm, Spodoptera littoralis (Lepidoptera: Noctuidae)

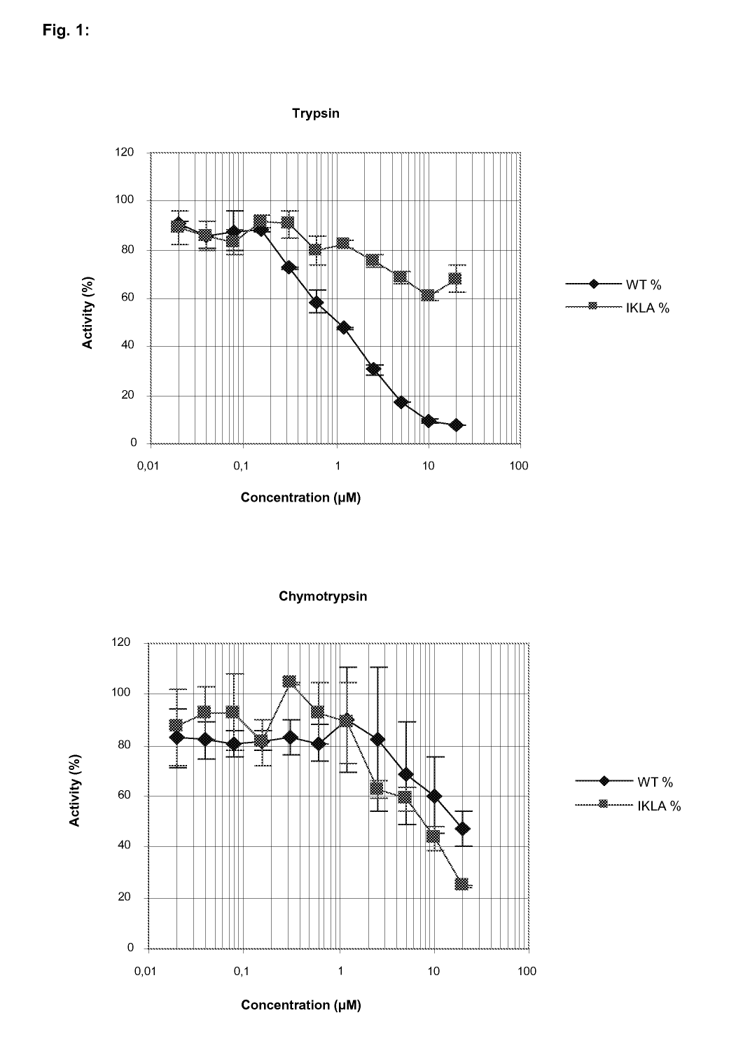

As shown in FIG. 1, AtSerpin1 exerts a clear inhibition of serine-like trypsin activities already at low concentration with 50% inhibition at about 1 μM concentrations. Maximal inhibition of trypsin activity was scored with ≧10 μM. In contrast, the IKLA (SEQ ID NO:3) mutant did not show potency to inhibit the trypsin protease activities of S. littoralis. Here, fluorometric substrate was employed to measure trypsin activities.

For chymotrypsin activity inhibition, the concentration of AtSerpin1 needed to inhibit 50% was estimated at 10 μM to 20 μM (FIG. 1). For the IKLA-derived (SEQ ID NO:3) variant of AtSerpin1, its potency to inhibit trypsin activity was lower than the original AtSerpin1, but its activity against chymotrypsin was similar.

With the use of colorimetric substrates, Table 1 confirms the high potency of AtSerpin1 to inhibit trypsin protease activities from S. littoralis extracts with 52% inhibition at 1.25 μM and 83% at 5 μM. With the three different derived variants (mutant IKLA (SEQ ID NO:3), IKLK (SEQ ID NO:7), and VRPR (SEQ ID NO:4)), the activity was lower compared to AtSerpin1, and for the mutants WRHR (SEQ ID NO:6) and IRPR (SEQ ID NO:5), there was no activity present. The tests for inhibition of chymotrypsin activities with AtSerpin1 and the IKLA (SEQ ID NO:3) mutant showed activity with 40% to 56% inhibition with the highest concentration tested (10 μM).

TABLE 1
Inhibition of serine-like protease activities from extracts of larvae of
the cotton leafworm, Spodoptera littoralis (Lepidoptera), by AtSerpin1
and derived mutants.

| | % Inhibition | | | | | |
|---|---|---|---|---|---|---|
| | Trypsin | | | Chymotrypsin | | |
| | 1.25 μM | 5 μM | 10 μM | 1.25 μM | 5 μM | 10 μM |
| AtSerpin1 | 52 ± 1 | 83 ± 1 | 90 ± 1 | 10 ± 21 | 31 ± 20 | 40 ± 15 |
| Mutant IKLA | 18 ± 1 | 32 ± 2 | 40 ± 2 | 11 ± 16 | 41 ± 5 | 56 ± 5 |
| (SEQ ID NO: 3) | | | | | | |
| Mutant IKLK | — | 64 ± 2 | 69 ± 3 | — | — | — |
| (SEQ ID NO: 7) | | | | | | |
| Mutant VRPR | ni | 33 ± 1 | 34 ± 3 | ni | — | — |
| (SEQ ID NO: 4) | | | | | | |
| Mutant WRHR | ni | — | — | ni | — | — |
| (SEQ ID NO: 6) | | | | | | |
| Mutant IRPR | ni | — | — | ni | — | — |
| (SEQ ID NO: 5) | | | | | | |

— = not determined
ni = no inhibition

### Example 2

### Inhibition of the Enzymes of the Mediterranean Corn Borer, Sesamia nonagrioides (Lepidoptera: Noctuidae)

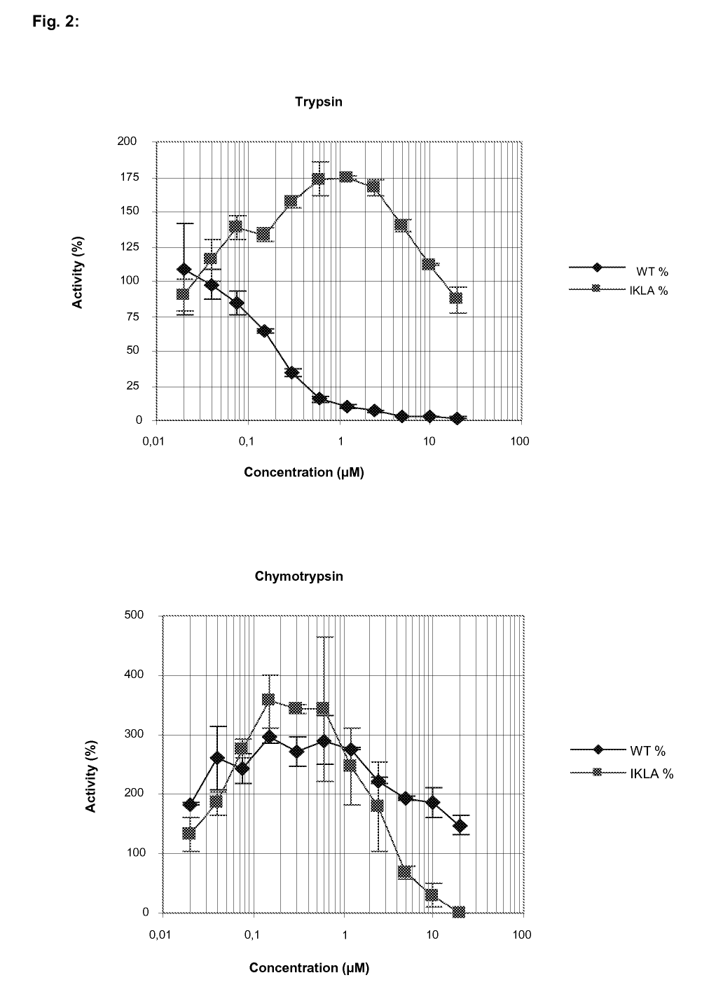

FIG. 2 demonstrates a very strong inhibition by AtSerpin1 of trypsin activities already at low concentration with 50% inhibition at about 0.1 μM concentrations. Maximal inhibition of trypsin activity was scored already with 0.5 μM to 1 μM. The IKLA-derived (SEQ ID NO:3) variant scored low inhibitory activity.

For chymotrypsin activity inhibition, the concentration of AtSerpin1 needed to inhibit ≧50% was estimated at 0 μM. Interestingly, for the IKLA-derived (SEQ ID NO:3) variant of AtSerpin1, its potency to inhibit chymotrypsin activity was higher than the original AtSerpin1.

Table 2 clearly provides confirmation of the high potency of AtSerpin1 to inhibit trypsin protease activities from S. nonagrioides extracts. With the derived IKLA (SEQ ID NO:3) variant, the activity was lost. The tests for inhibition of chymotrypsin activities with AtSerpin1 showed no activity; however with the mutant IKLA (SEQ ID NO:3), a high activity with 70% inhibition was scored at 10 μM.

TABLE 2
Inhibition of serine-like protease activities from extracts of larvae of
the Mediterranean corn borer, Sesamia nonagrioides (Lepidoptera),
by AtSerpin1 and derived mutants.

| | % Inhibition | | | | | |
|---|---|---|---|---|---|---|
| | Trypsin | | | Chymotrypsin | | |
| | 1.25 | | | 1.25 | | |
| | μM | 5 μM | 10 μM | μM | 5 μM | 10 μM |
| AtSerpin1 | 90 ± 1 | 97 ± 1 | 98 ± 1 | ni | ni | ni |
| Mutant IKLA | ni | ni | ni | ni | 32 ± 10 | 70 ± 20 |
| (SEQ ID NO: 3) | | | | | | |
| Mutant WRHR | ni | — | — | ni | — | — |
| (SEQ ID NO: 6) | | | | | | |
| Mutant IRPR | 38 ± 6 | — | — | ni | — | — |
| (SEQ ID NO: 5) | | | | | | |
| Mutant VRPR | ni | — | — | ni | — | — |
| (SEQ ID NO: 4) | | | | | | |

ni = no inhibition
— = not determined

### Example 3

### Inhibition of the Enzymes of the European Corn Borer, Ostrinia nubilalis (Lepidoptera: Crambidae)

With use of colorimetric substrates for trypsin and chymotrypsin activities, AtSerpin1 shows limited potency to inhibit trypsin protease activities from O. nubilalis extracts with 28% at 2 μM (Table 3). With the derived IKLA (SEQ ID NO:3) variant, the activity was much higher reaching 51% inhibition at 2 μM. The tests for inhibition of chymotrypsin activities with AtSerpin1 showed no activity, whereas the mutant IKLA (SEQ ID NO:3) exerted 21% inhibition at 2 μM.

TABLE 3
Inhibition of serine-like protease activities from
O. nubilalis extracts by AtSerpin1 and its
derived variant IKLA (SEQ ID NO: 3).

% Inhibition

| | Trypsin | Elastase |
|---|---|---|
| | 2 μM | 2 μM |
| AtSerpin1 | 28 ± 2 | ni |
| Mutant IKLA (SEQ ID NO: 3) | 51 ± 1 | 21 ± 1 |

ni = no inhibition

### Example 4

### Inhibition of the Enzymes of the Colorado Potato Beetle, Leptinotarsa decemlineata (Coleoptera: Chrysomelidae)

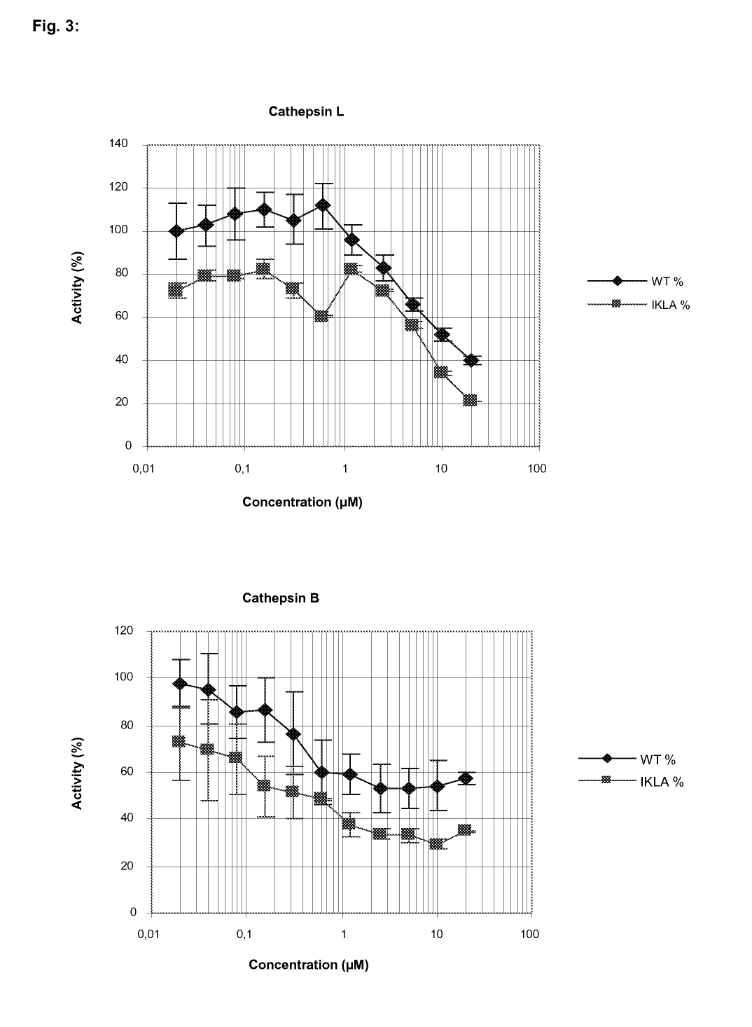

As shown in FIG. 3 with use of fluorometric substrates, AtSerpin1 provokes 50% inhibition of cysteine-like cathepsin-L protease activities at 10 μM in the Colorado potato beetle gut extracts.

For the catherpsin-B activity inhibition, the concentration of AtSerpin1 needed to inhibit 50% was estimated >10 μM. For the IKLA-derived (SEQ ID NO:3) variant of At Serpin1, its potency to inhibit both cysteine-like protease activities, cathepsin-B and cathepsin-Lactivity, was higher than the original AtSerpin1 (FIG. 3).

With use of colorimetric substrates, AtSerpin1 inhibits cathepsin-B protease activities with 40% already at relatively low concentrations of 1.25 μM (Table 4). For cathepsin-L activities, the potency of AtSerpin 1 for inhibition was lower, yielding 4% at 1.25 μM and increasing up to 48% at 10 μM. Interestingly, with the derived variant IKLA (SEQ ID NO:3), the activity was higher as with the original AtSerpin1 (Table 4). With the lowest concentration tested (1.25 μM) already, 62% inhibition of cathepsin-B was scored. The tests for inhibition of cathepsin-L activities with IKLA (SEQ ID NO:3) scored 66% at 10 μM.

TABLE 4
Inhibition of cysteine-like protease activities from extracts of the
Colorado potato beetle, Leptinotarsa decemlineata (Coleoptera),
by the AtSerpin1 and derived mutant IKLA (SEQ ID NO: 3).

| | % Inhibition | | | | | |
|---|---|---|---|---|---|---|
| | Cathepsin-B | | | Cathepsin-L | | |
| | 1.25 μM | 5 μM | 10 μM | 1.25 μM | 5 μM | 10 μM |
| AtSerpin1 | 40 ± 8 | 47 ± 8 | 46 ± 11 | 4 ± 10 | 34 ± 4 | 48 ± 4 |
| Mutant | 62 ± 5 | 77 ± 3 | 70 ± 2 | 18 ± 2 | 44 ± 2 | 66 ± 1 |
| IKLA | | | | | | |
| (SEQ ID | | | | | | |
| NO: 3) | | | | | | |

TABLE 5
Inhibition of cysteine-like protease activities (cathepsin-L) from extracts of adults of the red flour beetle,
Tribolium castaneum (Coleoptera), by AtSerpin1 and derived mutants.

| | % Inhibition | | | | | | | | | |
|---|---|---|---|---|---|---|---|---|---|---|
| | Cathepsin-L | | | | | | | | | |
| | 0.04 μM | 0.08 μM | 0.16 μM | 0.31 μM | 0.63 μM | 1.25 μM | 2.5 μM | 5 μM | 10 μM | 20 μM |
| AtSerpin1 | ni | ni | ni | ni | ni | ni | 16 ± 1 | 68 ± 1 | 73 ± 8 | 76 ± 12 |
| Mutant IKLA (SEQ ID | ni | 12 ± 1 | 32 ± 4 | 57 ± 5 | 63 ± 2 | 68 ± 9 | 68 ± 7 | 72 ± 17 | 70 ± 8 | 76 ± 15 |
| NO: 3) | | | | | | | | | | |
| Mutant WRHR (SEQ ID | — | — | — | — | — | ni | — | — | — | — |
| NO: 6) | | | | | | | | | | |
| Mutant IRPR (SEQ ID | — | — | — | — | — | 73 ± 1 | — | — | — | — |
| NO: 5) | | | | | | | | | | |
| Mutant VRPR (SEQ ID | — | — | — | — | — | 38 ± 2 | — | — | — | — |
| NO: 4) | | | | | | | | | | |

ni = no inhibition
— = not determined

### Example 5

### Inhibition of the Enzymes of the Red Flour Beetle, Tribolium castaneum (Coleoptera: Tenebrionidae)

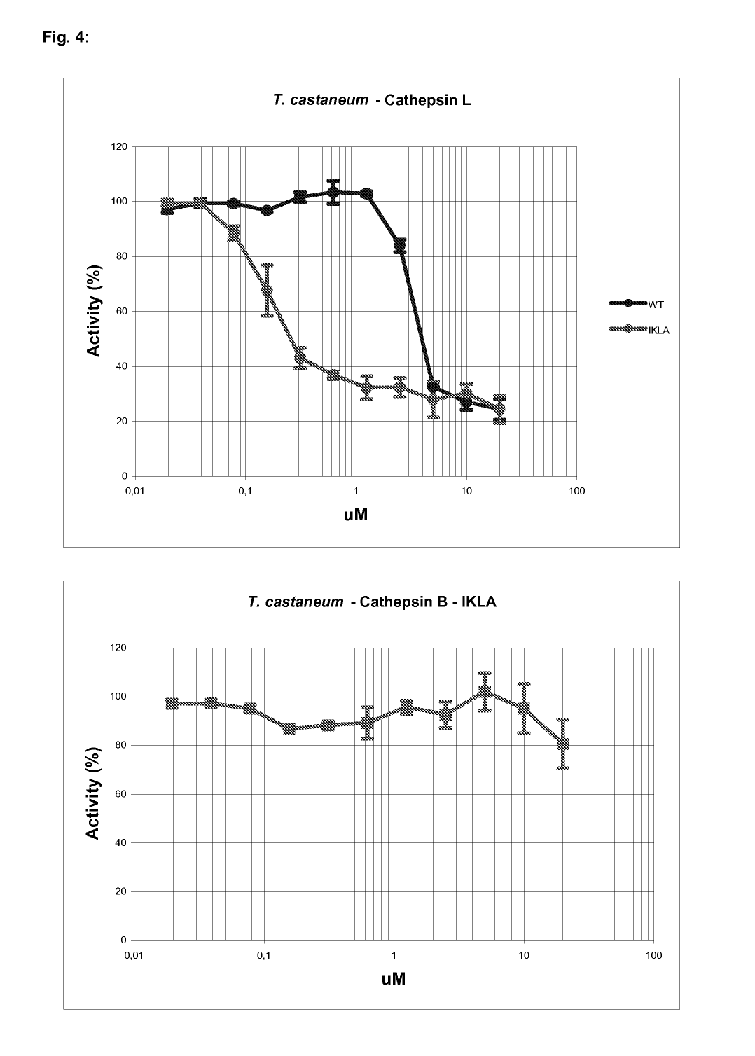

FIG. 4 shows that the cysteine-like cathepsin-L activity is inhibited by the AtSerpin1 with an IC50=4 μM. Very interestingly, the IKLA (SEQ ID NO:3) mutant is >10 times more active and the estimated IC50 yielded 0.2 μM to 0.3 μM to inhibit 50% of cathepsin-L activity. For the cathepsin-B activities, no inhibitory activity was scored with IKLA-derived (SEQ ID NO:3) mutant of AtSerpin1 when tested up to 20 μM. The results with the other mutant serpins are summarized in Table 5.

### Example 6

### Inhibition of the Enzymes of the Pea Aphid, Acyrthosiphon pisum (Homoptera: Aphididae)

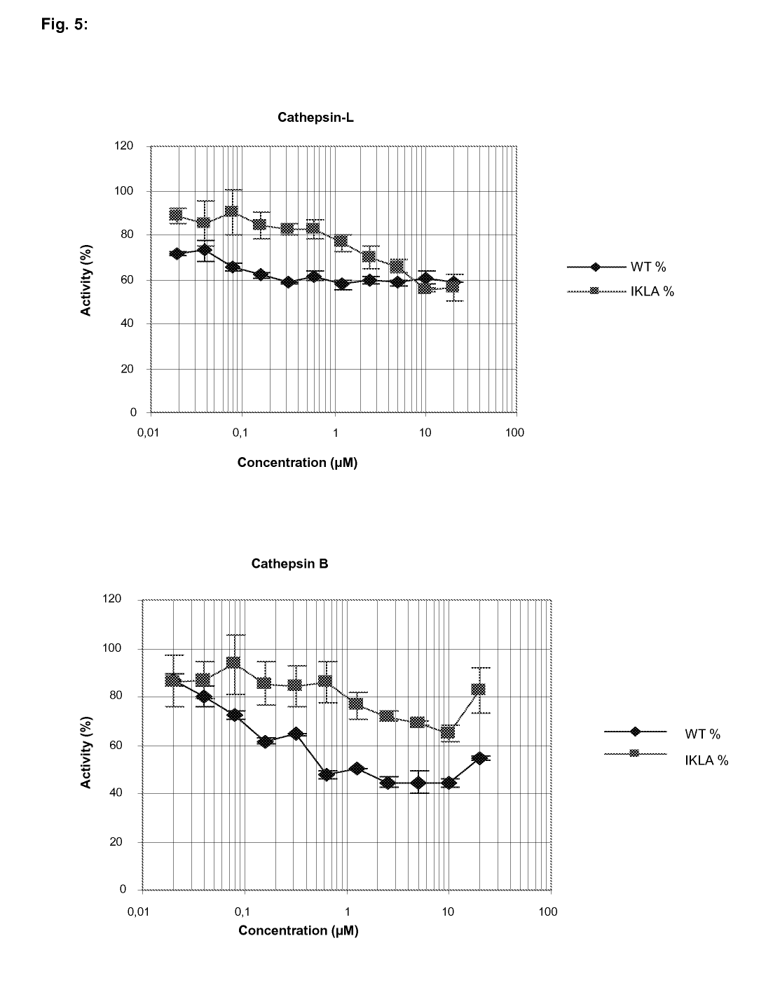

As shown in FIG. 5, AtSerpin1 exerts inhibition of cysteine-like cathepsin-L protease activities. However, it was strange in this experiment that the effect with AtSerpin1 flattened around 40% inhibition. Otherwise, it was clear that the inhibitory activity of AtSerpin1 was stronger than for the IKLA (SEQ ID NO:3) mutant.

For the cysteine-like cathepsin-B activity inhibition of aphid A. pisum extracts, the concentration of AtSerpin1 needed to inhibit 50% was estimated at around 1 μM (FIG. 5). For the IKLA-derived (SEQ ID NO:3) variant of AtSerpin1, its potency to inhibit cathepsin-B activity was lower than the original AtSerpin1.

With use of colorimetric substrates, Table 6 confirms the high potency of AtSerpin1 to inhibit cathepsin-B protease activities from A. pisum aphid extracts. 50% inhibition of cathepsin-B was scored with relatively low concentrations of 1.25 μM. For cathepsin-L activities, the percentage of inhibition was somewhat lower yielding 39% to 42% at 1.25 μM to 10 μM.

As shown in Table 6, with the derived variant IKLK (SEQ ID NO:7), the activity was similar as with the original AtSerpin1, whereas with IKLA (SEQ ID NO:3) and VRPR (SEQ ID NO:4), the activities were lower. The tests for inhibition of cathepsin-L activities with IKLA (SEQ ID NO:3) scored somewhat similar as AtSerpin1.

TABLE 6
Inhibition of cysteine-like protease activities from extracts of adults of
the pea aphid, Acyrthosiphon pisum (Homoptera), by AtSerpin1
and derived mutants.

| | % Inhibition | | | | | |
|---|---|---|---|---|---|---|
| | Cathepsin-B | | | Cathepsin-L | | |
| | 1.25 | | | 1.25 | | |
| | μM | 5 μM | 10 μM | μM | 5 μM | 10 μM |
| AtSerpin1 | 50 ± 1 | 55 ± 5 | 55 ± 2 | 42 ± 3 | 42 ± 2 | 39 ± 4 |
| Mutant IKLA | 24 ± 6 | 31 ± 1 | 36 ± 3 | 24 ± 6 | 34 ± 4 | 45 ± 1 |
| (SEQ ID | | | | | | |
| NO: 3) | | | | | | |
| Mutant IKLK | — | 49 ± 4 | 66 ± 4 | — | — | — |
| (SEQ ID | | | | | | |
| NO: 7) | | | | | | |
| Mutant VRPR | 27 ± 1 | 14 ± 2 | 23 ± 1 | 16 ± 1 | — | — |
| (SEQ ID | | | | | | |
| NO: 4) | | | | | | |
| Mutant WRHR | 26 ± 2 | — | — | ni | — | — |
| (SEQ ID | | | | | | |
| NO: 6) | | | | | | |
| Mutant IRPR | 32 ± 2 | — | — | 31 ± 2 | — | — |
| (SEQ ID | | | | | | |
| NO: 5) | | | | | | |

ni = no inhibition
— = not determined

### Example 7

### Inhibition of the Enzymes of the Sweetpotato Whitefly, Bemisia tabaci (Homoptera: Aleyrodidae)

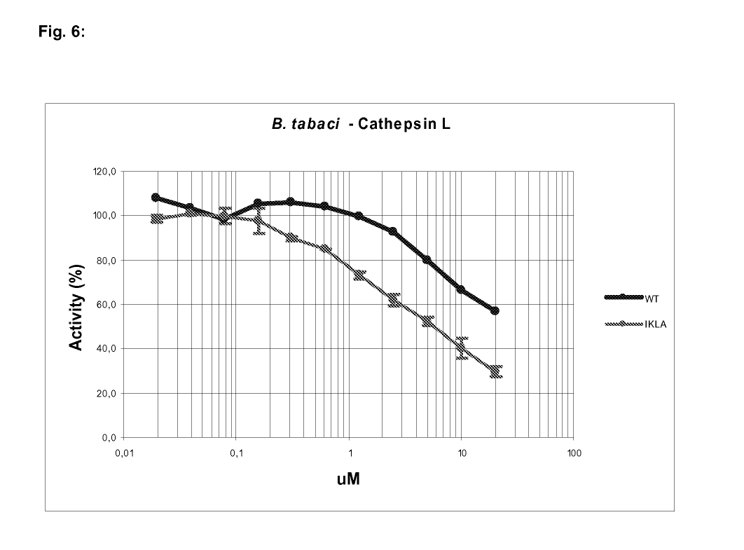

FIG. 6 demonstrates that the cysteine-like cathepsin-L activity is inhibited by the AtSerpin1 with the highest concentration tested (20 μM) 45% inhibition was scored. Very interestingly, the IKLA (SEQ ID NO:3) mutant is about five to ten times more active and the estimated IC50 is around 5 μM to inhibit 50% of cathepsin-L activity. The results are further summarized in Table 7.

### Example 8

### Spider Mites, Tetranychus urticae (Acari: Tetranychidae)

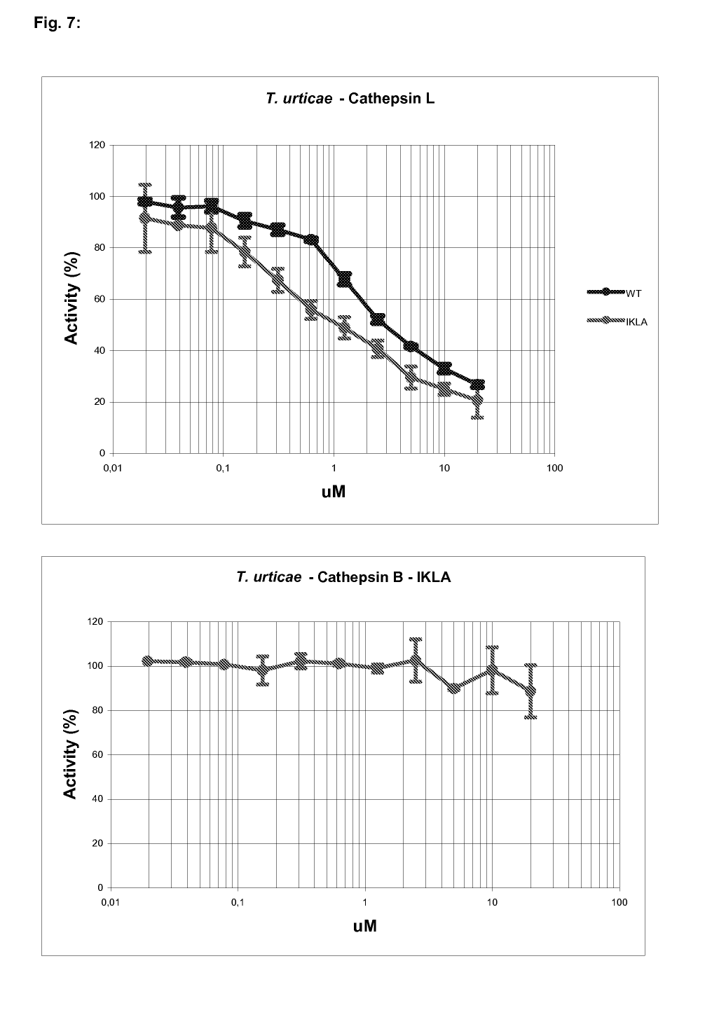

In spider mites, AtSerpin1 is active to inhibit cathepsin-L protease activities with 50% inhibition at about 2 μM (FIG. 7). Very interesting, the IKLA-derived (SEQ ID NO:3) mutant of AtSerpin1 is even more active with an IC50<1 μM; which is two to three times higher in activity. For the cathepsin-B activities, no inhibitory activity was scored with IKLA-derived (SEQ ID NO:3) mutant of AtSerpin1 when tested up to 20 μM and also with WRHR (SEQ ID NO:6), VRPR (SEQ ID NO:4), IRPR-derived (SEQ ID NO:5) mutant of AtSerpin1 when tested at 1 μM.

The results for the IKLA (SEQ ID NO:3) mutant, in comparison with AtSerpin1 and other mutants (at a concentration of 1.25 μM) are summarized in Table 8.

### Example 9

### Inhibition of Growth and Development, and Mortality in Pea Aphid Acyrthosiphon pisum Fed on a Diet Comprising AtSerpin1 Mutant IKLA (SEQ ID NO:3).

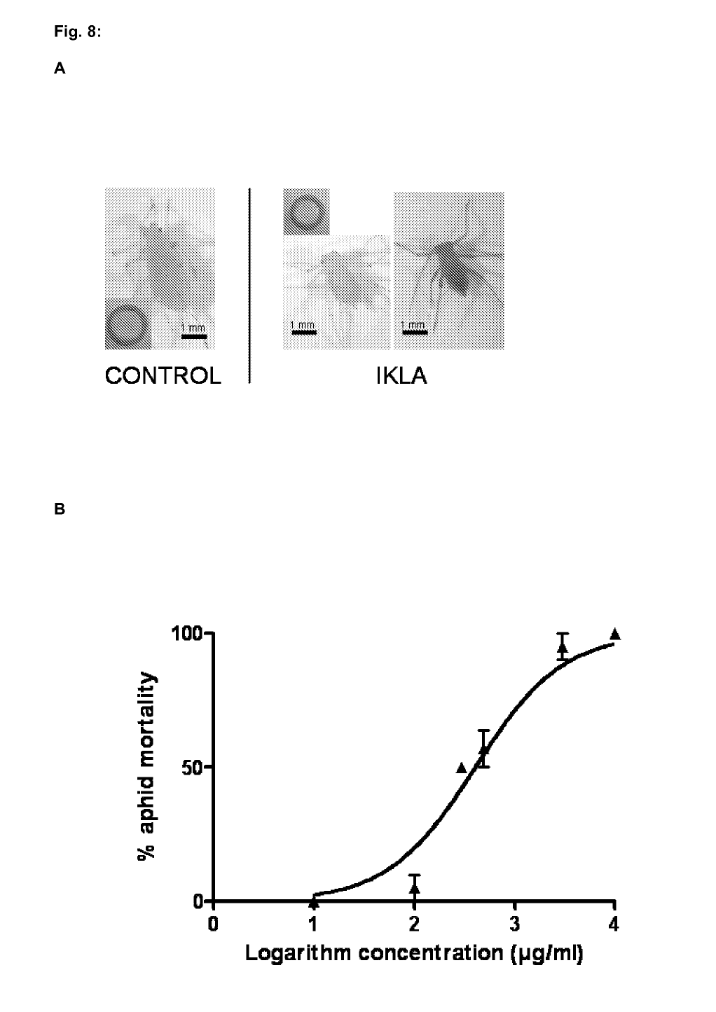

Inhibition of aphid nymphal growth and development was measured on a diet comprising recombinant AtSerpin1 mutant IKLA (SEQ ID NO:3) as compared to an artificial control diet where the nymphal growth/development was normal. The inhibitory effects were already visible with concentrations of 500 μg/ml. In these affected nymphs, a conspicuous inhibition of size increase can be seen already after two to three days of treatment. As exemplified in FIG. 8A, the intoxicated aphids show an average reduction of 50% and then this was followed by death of these individuals; the dead aphids color brown. The median lethal concentration (LC50) of IKLA (SEQ ID NO:3) protein in diet was estimated to be 406 μg/ml (95% confidence interval: 271-607 μg/ml; R2=0.95) (FIG. 8B).

TABLE 7
Inhibition of cysteine-like protease activities (cathepsin-L) from extracts of adults of the sweetpotato
whitefly, Bemisia tabaci (Homoptera) by AtSerpin1 and derived mutant IKLA (SEQ ID NO: 3).

| | % Inhibition | | | | | | | | | |
|---|---|---|---|---|---|---|---|---|---|---|
| | Cathepsin-L | | | | | | | | | |
| | 0.04 μM | 0.08 μM | 0.16 μM | 0.31 μM | 0.63 μM | 1.25 μM | 2.5 μM | 5 μM | 10 μM | 20 μM |
| AtSerpin1 | ni | ni | ni | ni | ni | ni | 7 ± 1 | 20 ± 1 | 34 ± 1 | 43 ± 1 |
| Mutant IKLA | ni | ni | 3 ± 1 | 10 ± 1 | 15 ± 0 | 27 ± 1 | 38 ± 2 | 48 ± 2 | 60 ± 7 | 70 ± 5 |
| (SEQ ID NO: 3) | | | | | | | | | | |

ni = no inhibition
— = not determined

TABLE 8
Inhibition of cysteine-like protease activities (cathepsin-L) from extracts of adults of the spider mites,
Tetranychus urticae (Acari) by AtSerpin1 and derived mutants.

| | % Inhibition | | | | | | | | | | |
|---|---|---|---|---|---|---|---|---|---|---|---|
| | Cathepsin-L | | | | | | | | | | |
| | 0.02 | 0.04 | | | | | | | | | |
| | μM | μM | 0.08 μM | 0.16 μM | 0.31 μM | 0.63 μM | 1.25 μM | 2.5 μM | 5 μM | 10 μM | 20 μM |
| AtSerpin1 | ni | ni | ni | 10 ± 1 | 13 ± 1 | 17 ± 1 | 32 ± 1 | 48 ± 1 | 58 ± 1 | 67 ± 3 | 73 ± 3 |
| Mutant IKLA | 9 ± 1 | 11 ± 1 | 12 ± 1 | 22 ± 2 | 33 ± 2 | 44 ± 3 | 51 ± 4 | 59 ± 5 | 71 ± 10 | 75 ± 6 | 79 ± 26 |
| (SEQ ID NO: 3) | | | | | | | | | | | |
| Mutant VRPR | — | — | — | — | — | — | 22 ± 3 | — | — | — | — |
| (SEQ ID NO: 4) | | | | | | | | | | | |
| Mutant WRHR | — | — | — | — | — | — | 8 ± 1 | — | — | — | — |
| (SEQ ID NO: 6) | | | | | | | | | | | |
| Mutant IRPR | — | — | — | — | — | — | 11 ± 1 | — | — | — | — |
| (SEQ ID NO: 5) | | | | | | | | | | | |

ni = no inhibition
— = not determined

### REFERENCES

Alford D. V. (2000). Pest and disease management handbook. British Crop Protection Council, Blackwell Science, Oxford, 615 pp.
Brandt A., I. Svendsen, and J. Hejgaard (1990). A plant serpin gene. Structure, organisation and expression of the gene encoding barley protein Z. Eur. J. Biochem. 194, 499-505.
Dahl S., S. W. Rasmussen, and J. Hejgaard (1996). Heterologous expression of three plant serpins with distinct inhibitory specificities. J. Biol. Chem. 271, 25083-25088.
Dulmage H. T. (1981). Production of bacteria for biological control of insects. In “Biological control in crop production,” D. C. Paparrizas, ed., pp. 129-141.
Febvay G., B. Delobel, and Y. Rahbé (1988). Influence of the amino-acid balance on the improvement of an artificial diet for a biotype of Acyrthosiphon pisum (Homoptera, Aphididae). Can. J. Zool. 66, 2449-2453.
Girard C., M. Le Metayer, B. Zaccomer, B. Barlet, E. Barlet, I. Williams, M. Bonade-Bottino, M. H. Pham-Delegue, and L. Jouanin (1998). Growth stimulation of beetle larvae reared on a transgenic oilseed rape expressing a cysteine proteinase inhibitor. J. Insect Physiol. 44, 263-270.
Goldberg L. J. and J. Margalit (1977). A bacterial spore demonstrating rapid larvicidal activity against Anopheles segentii, Uranotaenia unguiculata, Culex univittatus, Aedes aegypti and Culex pipiens. Mosquito News 37, 355-358.
Hejgaard J. (1982). Purification and properties of protein Z—a major albumin of barley endosperm. Physiol. Plant 54, 174-182.
Hejgaard J. (2001). Inhibitory serpins from rye grain with glutamine as P1 and P2 residues in the reactive center. FEBS Lett. 488, 149-163.
Hejgaard J. and S. Hauge (2002). Serpins of oat (Avena sativa) grain with distinct reactive centres and inhibitory activity. Physiol. Plant 116, 155-163.
Haq S. K., S. M. Atif, and R. H. Khan (2004). Protein inhibitor genes in combat against insects, pest, and pathogens: natural and engineered phytoprotection. Arch. Biochem. Biophys. 431, 145-159.
Krieg A., A. M. Huger, and W. Schnetter (1987). Bacillus thuringiensis var san diego strain M-7 is identical to the formerly in Germany isolated strain BI 256-82, B. thuringiensis subsp. tenebrionis, which is pathogenic to coleopteran insects. Z. Angew. Ent. 104, 417-425.
Lungard R. and B. Svensson (1989). A 39 kD barley seed protein of the serpin superfamily inhibits alpha-chymotrypsin. Carlsberg Res. Commun. 54, 173-180.
Mathavan S., A. Velpandi, and J. C. Johnson (1987). Sub toxic effects of Bacillus spaericus 1593 M on feeding growth and reproduction of Laccotrephes griseus (Hemiptera: Nepidae). Exp. Biol. 46, 149-153.
Østergaard H., S. K. Rasmussen, T. H. Roberts, and J. Hejgaard (2000). Inhibitory serpins from wheat grain with reactive centers resembling glutamine-rich repeats of prolamin storage proteins. Cloning and characterization of five major molecular forms. J. Biol. Chem. 275, 33272-33279.
Patston P. A. (2000). Serpins and other serine protease inhibitors. Immunol. Today 21, 345-354.
Rasmussen S. K. (1993). A gene coding for a new plant serpin. Biochim. Biophys. Acta 1172, 151-154.
Rasmussen S. K., S. W. Dahl, A. Nørgard, and J. Hejgaard (1996). A recombinant wheat serpin with inhibitory activity. Plant Mol. Biol. 30, 673-677.
Roberts T. H., S. Martilla, S. K. Rasmussen, and J. Hejgaard (2003). Differential gene expression for suicide substrate serine proteinase inhibitors (serpins) in vegetative and grain tissues of barley. J. Exp. Bot. 54, 2251-2263.
Rosenkrands I., J. Heygaard, S. K. Rasmussen, and S. E. Bjorn (1994). Serpins from wheat grain, FEBS Lett. 343, 75-80.
Smagghe G. and D. Degheele (1994). Action of a novel nonsteroidal ecdysteroid mimic, tebufenozide (RH-5992), on insects of different orders. Pestic. Sci. 42, 85-92.
Smagghe G., L. Decombel, B. Carton, B. Voigt, G. Adam, and L. Tiny (2002). Action of brassinosteroids in the cotton leafworm Spodoptera littoralis. Insect Biochem. Molec. Biol. 32, 199-204.
Van Gent D., P. Sharp, K. Morgan, and N. Kalsheker (2003). Serpins: structure, function and molecular evolution. Int. J. Biochem. Cell. Biol. 35, 1536-1547.
Vercammen D., B. Belenghi, B. van de Cotte, T. Beunens, J. A. Gavigan, R. De Rycke, A. Brackenier, D. Inzé, J. L. Harris, and F. Van Breusegem (2006). Serpin 1 of Arabidopsis thaliana is a suicide inhibitor for metacaspase 9. J. Mol. Biol.
Yoo B. C., K. Aoki, Y. Xiang, L. R. Campbell, R. J. Hull, B. Xoconostle-Cazares, J. Monzer, J. Y. Lee, D. E. Ullman, and W. J. Lucas (2000). Characterization of Cucurbita maxima Phloem Serpin-1 (CmPS-1). A developmentally regulated elastase inhibitor. J. Biol. Chem. 275, 35122-35128.

What is claimed is:

1. A method for limiting insect-provoked and/or mite-provoked damage in a plant, wherein the improvement comprises:
utilizing a recombinant serpin, comprising a mutant reactive center loop for limiting insect and/or mite provoked damage in the plant.

2. The method according to claim 1, wherein said recombinant serpin is selected from the group consisting of Arabidopsis thaliana serpin At1g47710, Barley serpin BSZx, Barley serpin BSZ7, Wheat serpin WSZ2b, Cucumis sativus serpin gi|58416137, Oryza sativa serpin gi|37700305, Citrus x paradise serpin gi|26224736, Solanum tuberosum serpin gi|62950609, Lycopersicon esculentum serpin gi|14685955, Nicotiana tabacum serpin gi/52837819, Brassica rapa serpin gi/54416864, Vitis vinifera serpin gi|33401535, Antirrhinus majus serpin gi|51113970, Triphysaria versicolor serpin gi|68033793, Helianthus paradoxus serpin gi|33123072, Gossypium arboreturn serpin gi|13245345, Populus nigra serpin gi/60218046 and Brassica napus serpin (SEQ ID NO: 2).

3. The method according to claim 2, wherein said recombinant serpin is derived from SEQ ID NO: 1 (At1g47710)

4. A method for limiting insect-provoked and/or mite-provoked damage in a plant, wherein the improvement comprises:
utilizing a recombinant serpin, comprising a reactive center loop comprising a sequence selected from the group consisting of IKLA (SEQ ID NO:3), VRPR (SEQ ID NO:4), IRPR (SEQ ID NO:5) WRHR (SEQ ID NO:6), and IKLK (SEQ ID NO:7) for limiting insect and/or mite provoked damage in plants.

5. The method according to claim 1, wherein said insect and/or mite provoked damage is caused by a chewing insect.

6. The method according to claim 5, wherein said insect or mite belongs to an order selected from the group consisting of the orders Lepidoptera, Coleoptera, Homoptera and Acari.

7. The method according to claim 6, wherein said insect or mite is selected from the group consisting of Spodoptera spp, Sesamia spp., Ostrinia spp., Leptinotarsa spp., Tribolium spp., Acyrthosiphon spp., Bemisia spp. and Tetranychus spp.

8. The method according to claim 1, wherein said recombinant serpin is applied by spraying.

9. The method according to claim 1-6, wherein said serpin is overexpressed in a plant.

10. A method to develop new mutant serpins with a specific inhibition spectrum, the method comprising mutating one or more amino acids of the reactive center loop of a serpin.

11. A transgenic plant, expressing a recombinant serpin, wherein the recombinant serpin has a mutant reactive center loop.

12. The transgenic plant of claim 11, wherein said recombinant serpin comprises a reactive center loop comprising a sequence selected from the group consisting of IKLA (SEQ ID NO:3), VRPR (SEQ ID NO:4), IRPR (SEQ ID NO:5), WRHR (SEQ ID NO:6), and IKLK (SEQ ID NO:7).
